(19) United States
(12) Patent Application Publication
(10) Pub. No.: US 2026/0308179 A1
(43) Pub. Date: Oct. 8, 2026

(54) NOVEL CRYSTALLINE FORM OF RISDIPLAM AND PROCESS FOR PREPARATION THEREOF

(71) Applicant: HARMAN FINOCHEM LIMITED, Mumbai, Maharashtra (IN)

(72) Inventors: Nitin Sharadchandra PRADHAN, Thane (West), Maharashtra (IN); Vihar Raghunath TELANGE, Thane, Maharashtra (IN); Yuvaraj Suresh SONAR, Thane, Maharashtra (IN); Vaibhav Chandrakant SHINDE, Navi Mumbai, Maharashtra (IN); Harpreet Singh MINHAS, Mumbai, Maharashtra (IN); Gurpreet Singh MINHAS, Mumbai, Maharashtra (IN)

(21) Appl. No.: 19/481,701

(22) PCT Filed: Apr. 30, 2024
(86) PCT No.: PCT/IN2024/050457

§ 371 Date: Nov. 5, 2025

(30) Foreign Application Priority Data
May 13, 2023 (IN) 202321033768

(51) Int. Cl. C07D 519/00

(52) U.S. Cl. CPC C07D 519/00

(57) ABSTRACT

The present invention discloses a novel crystalline form of Risdiplam of structural formula-1 and process for preparation thereof. The present invention further discloses pharmaceutical composition comprising the said crystalline form and their use as pharmaceutically active compounds. Particularly, the present invention relates to novel crystalline Risdiplam polymorph Form H, process for preparation and pharmaceutical composition thereof.

### FIELD OF THE INVENTION

The present invention relates to a novel crystalline form of Risdiplam of structural formula-1 and process for preparation thereof. The present invention further relates to pharmaceutical composition comprising the said crystalline form and their use as pharmaceutically active compounds. Particularly, the present invention relates to novel crystalline Risdiplam polymorph Form H, process for preparation and pharmaceutical composition thereof.

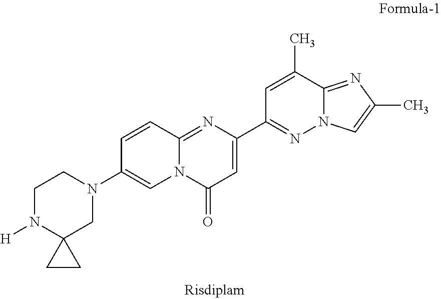 [SMILES: Cc1cn2nc(-c3cc(=O)n4cc(N5CCNC6(CC6)C5)ccc4n3)cc(C)c2n1]

### BACKGROUND OF THE INVENTION

7-(4,7-Diazaspiro[2,5]octan-7-yl)-2-(2,8-dimethylimidazo[1,2-b]pyridazin-6-yl)-pyrido[1,2-a]pyrimidin-4-one (Formula-1) is commonly known as Risdiplam. Risdiplam is a medication used to treat spinal muscular atrophy (SMA) and the first oral medication approved to treat this disease. Risdiplam is approved by the US Food and Drug Administration (FDA) and by European Medicines Agency under the brand name of Evrysdi for the treatment of SMA in patients of 2 months of age and older.

U.S. Pat. No. 9,969,754B2 discloses Risdiplam and its process for the preparation thereof. However, this process results in Risdiplam very poor yields. The process is shown in below scheme 1. According to this process compound (a) is reacted with compound (b) in presence of Pd(Ph₃P)₄ to obtain 2-(2,8-dimethylimidazo[1,2-b]pyridazin-6-yl)-7-fluoro-pyrido[1,2-a]pyrimidin-4-one, which is reacted with 4,7-diazaspiro[2.5]octane hydrochloride to yield Risdiplam.

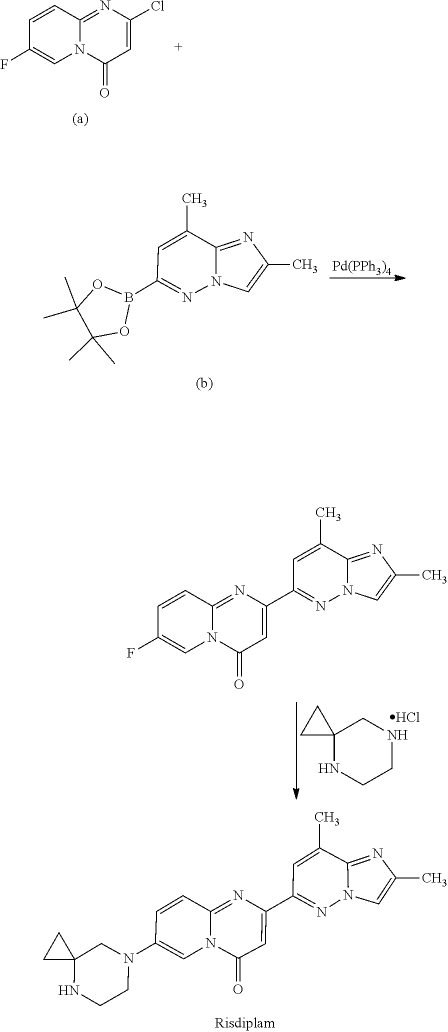

U.S. Pat. No. 11,390,632B2 disclosed the following route of synthesis of Risdiplam, as shown in scheme 2. According to this synthetic scheme, the compound of formula III reacted with compound of formula III′ in the presence of a palladium catalyst or nickel catalyst to form the compound of formula (II); and reacting the compound of formula (II) with a strong acid to obtain a compound of formula (I).

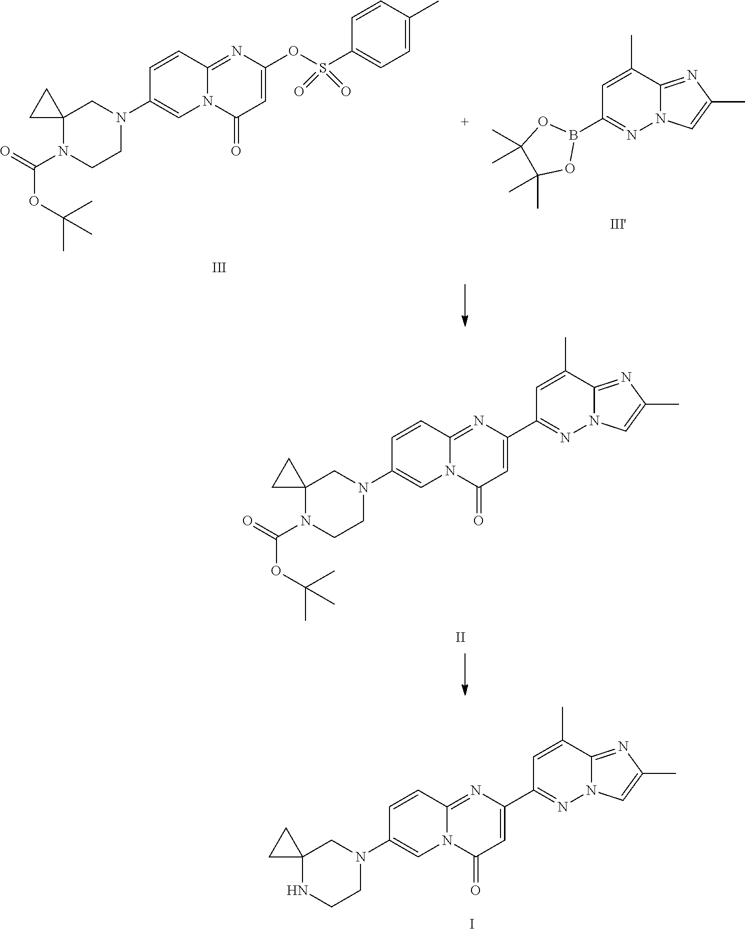

The processes described in the above prior arts resulted in poor yields and they require multiple chromatographic purifications for the isolation of Risdiplam and its intermediates which are considered to be very expensive for commercial scale preparations.

Further, WO2020079203 discloses the crystalline Form A of Risdiplam. WO2021021775 discloses crystalline form 1, form 2, form 3, form 4 and form 5 of Risdiplam. WO2022162107 describes the crystalline Form 1 of Risdiplam.

However, there is need in art to provide a novel crystalline form of Risdiplam with high purity and yield, to overcome the problems associated with the prior arts as described above.

### SUMMARY OF THE INVENTION

The primary object of the present invention is to provide a novel crystalline Form H of 7-(4,7-diazaspiro[2.5]octan-7-yl)-2-(2,8-dimethylimidazo[1,2-b]pyridazin-6-yl)pyrido-4H-[1,2-a]pyrimidin-4-one (Risdiplam) of structural formula-1 with high yield and purity.

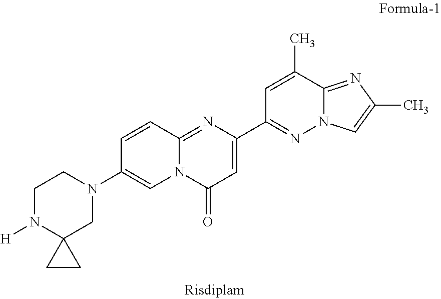 [SMILES: Cc1cn2nc(-c3cc(=O)n4cc(N5CCNC6(CC6)C5)ccc4n3)cc(C)c2n1]

In an aspect, the crystalline Form H of Risdiplam is characterized by a PXRD data having peaks at 5.94, 11.05, 15.99, 20.27, 23.59 2θ±0.2θ.

In another aspect, the crystalline Form H of Risdiplam comprises the additional PXRD peaks at 15.41, 17.23, 24.74, 26.53, 27.07 2θ±0.2θ. The PXRD peaks for Risdiplam Form H is illustrated in FIG. 1.

In yet another aspect, the crystalline Form H of Risdiplam is characterized by the IR spectrum at 834 cm⁻¹, 880 cm⁻¹, 926 cm⁻¹, 1210 cm⁻¹, 1227 cm⁻¹, 1674 cm⁻¹, 2824 cm⁻¹, 3283 cm⁻¹; as illustrated in FIGS. 2A and 2B. The IR spectrum of Risdiplam Form H was conducted after 6 months of storage.

In another aspect, the crystalline Form H of Risdiplam is characterized by the DSC comprising two endotherms (i) a broad endothermic peak at 242-248° C. and an endothermic peak with an onset at 280.55° C. and a peak at temperature of about 286° C. as illustrated in FIG. 3A-3C.

In an aspect, the crystalline Form H of the present invention is in anhydrous form having water content less than 2.0% as determined by thermogravimetric analysis (TGA) in table below.

In yet another aspect, the present invention provides a process for preparation of crystalline Form H of Risdiplam of structural formula-1.

Accordingly, the process for preparation of crystalline Form H of Risdiplam of structural formula-1, comprising:
a. Adding Risdiplam in a mixture of solvent(s);
b. Stirring at temperature ranging between 25 to 40° C.; and
c. Precipitating the crystalline solid obtained in step b) followed by filtering, washing and drying.

In an aspect, the said process for preparation of crystalline Form H of Risdiplam does not employ multiple chromatographic purifications for isolation.

In yet another aspect, the crystalline Form H of Risdiplam of structural formula-1 prepared according to the above process is in high yield, purity and is substantially free of chemical impurities.

In an aspect, the present invention provides a pharmaceutical composition comprising of crystalline Form H of Risdiplam prepared by the present process as an active ingredient together with pharmaceutically acceptable carrier.

### BRIEF DESCRIPTIONS OF THE DRAWINGS

FIG. 1 illustrates PXRD data of crystalline Form H of Risdiplam of structural formula-1

FIG. 2A illustrates infrared spectrum (IR) of crystalline Form H of Risdiplam of structural formula-1; and 2B illustrates infrared spectrum (IR) of crystalline Form H of Risdiplam after 6 month storage.

FIG. 3A-C illustrates differential scanning calorimetry (DSC) of Risdiplam of structural formula-1.

### DETAILED DESCRIPTION OF THE INVENTION

The present invention will now be explained in detail with reference to its preferred and optional embodiments but should not be construed to limit the scope of the invention.

The terms which are introduced in the complete specification retain those definitions throughout the description and the claims.

Accordingly, the term “Crystalline” and “Polymorphs” refer to the particular compound in the particular crystalline form that is distinct from another crystalline form but shares the same chemical formula. Further, the crystalline state is irrespective of whether the chemical compound is solvated or not.

With regards to the “Polymorphic Purity” the crystalline polymorph Form H of Risdiplam is substantially free of chemical impurities (generated during preparation of the polymorphs) and of other polymorphic crystalline forms. “Substantially free” of chemical impurities for the purposes of this invention means less than or equal to about 5% w/w of chemical impurities, preferably less than or equal to 3% w/w of chemical impurities, more preferably less than or equal to about 2% w/w of chemical impurities, most preferably less than or equal to about 1% w/w of chemical impurities. The term “purified” or “in purified form” of the polymorph Form refers to the product obtained by the present process of preparation and which does not employ multiple chromatographic purifications for isolation.

In an embodiment, the present invention is directed to a crystalline Form H of 7-(4,7-diazaspiro[2.5]octan-7-yl)-2-(2,8-dimethylimidazo[1,2-b]pyridazin-6-yl)pyrido-4H-[1,2-a]pyrimidin-4-one (Risdiplam) of structural formula-1 with high yield and purity.

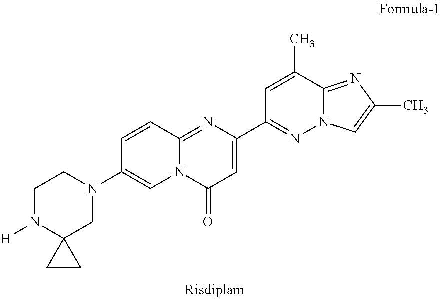 [SMILES: Cc1cn2nc(-c3cc(=O)n4cc(N5CCNC6(CC6)C5)ccc4n3)cc(C)c2n1]

In an embodiment, the crystalline Form H of Risdiplam is characterized by a PXRD data having peaks at 5.94, 11.05, 15.99, 20.27, 23.59 2θ±0.2θ.

In another embodiment, the crystalline Form H of Risdiplam comprises the additional PXRD peaks at 15.41, 17.23, 24.74, 26.53, 27.07 2θ±0.2θ.

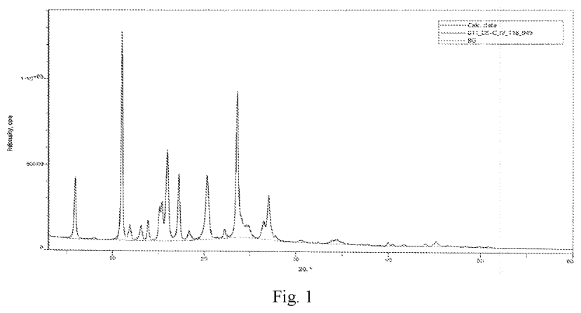

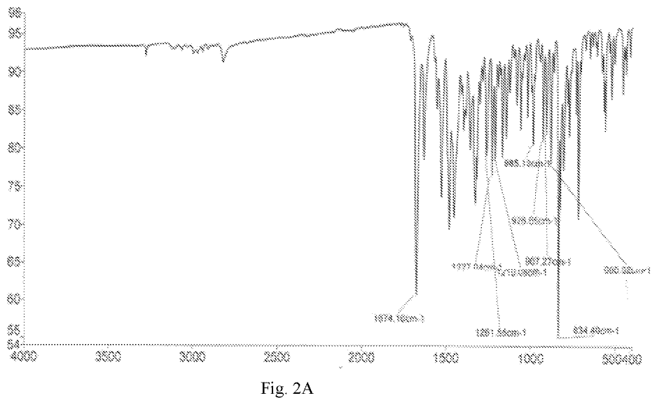

The PXRD peaks for Risdiplam Form His illustrated in FIG. 1.

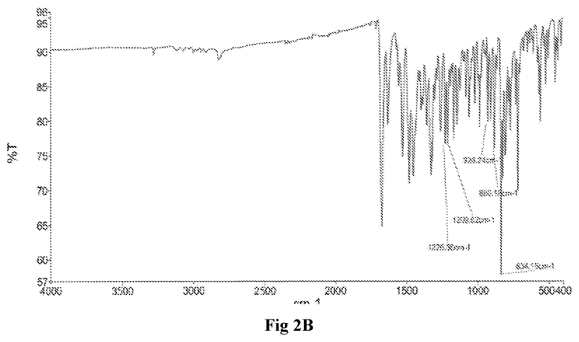

In yet another embodiment, the crystalline Form H of Risdiplam is characterized by the IR spectrum at 834 cm⁻¹, 880 cm⁻¹, 926 cm⁻¹, 1210 cm⁻¹, 1227 cm⁻¹, 1674 cm⁻¹, 2824 cm⁻¹, 3283 cm⁻¹ as illustrated in FIGS. 2A and 2B. The IR spectrum of Risdiplam Form H illustrated in FIG. 2B was conducted after 6 months of storage.

No change in the IR spectrum was observed indicating the stability of the crystalline Form H of Risdiplam.

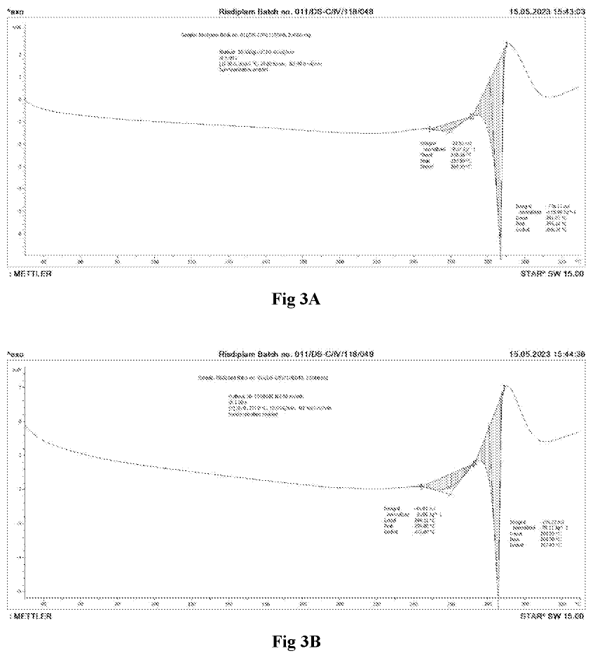

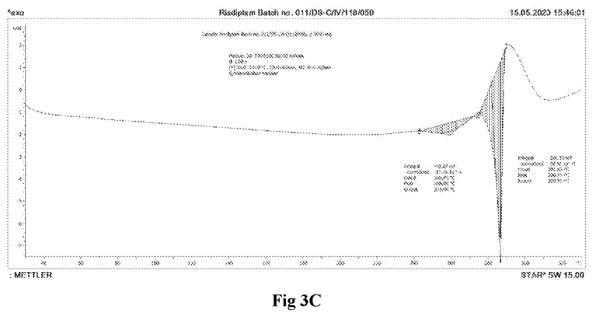

In another embodiment, the crystalline Form H of Risdiplam is characterized by the DSC comprising two endotherms (i) a broad endothermic peak at 242-248° C. and an endothermic peak with an onset at 280.55° C. and a peak at temperature of about 286° C. as illustrated in FIG. 3A-3C.

In an embodiment, the crystalline Form H of the present invention is in anhydrous form having water content less than 2.0% as determined by thermogravimetric analysis (TGA) shown in table below.

In yet another embodiment, the present invention discloses a process for preparation of crystalline Form H of Risdiplam of structural formula-1.

Accordingly, the process for preparation of crystalline Form H of Risdiplam having structural formula-1 comprises the steps of:
a. Adding Risdiplam in a mixture of solvent(s);
b. Stirring at a temperature ranging between 25 to 40° C.; and
c. Precipitating the crystalline solid obtained in step b) followed by filtering, washing and drying.

In an embodiment, the solvents are selected from polar, non-polar, protic or aprotic solvents selected from the groups consisting of alcohols, acids, esters hydrocarbons and such like alone or mixtures thereof.

The solvent for the process is particularly selected from lower C1-C4 alcohols, acids such as acetic acid, alone or mixtures thereof.

In an embodiment, the said process for preparation of crystalline Form H of Risdiplam does not employ multiple chromatographic purifications for isolation.

In an embodiment, the crystalline Form H of Risdiplam prepared according to the present invention is obtained in a yield of about more than 60%, preferably about more than 70% and more preferably about more than 80% and with the purity in the range of 99.3 to 99.7%.

In an embodiment, the crystalline Form H of Risdiplam prepared according to the present invention is characterized by a PXRD data having peaks at 5.94, 11.05, 15.99, 20.27, 23.59 2θ±0.2θ.

In another embodiment, the crystalline Form H of Risdiplam prepared by the present process comprises the additional PXRD peaks at 15.41, 17.23, 24.74, 26.53, 27.07 2θ±0.2θ as depicted in FIG. 1.

The crystalline Form H of Risdiplam prepared according to the present invention is characterized by an infrared spectrum (IR) having peaks at about 834 cm⁻¹, 880 cm⁻¹, 926 cm⁻¹, 1210 cm⁻¹ and 1227 cm⁻¹, 1674 cm⁻¹, 2824 cm⁻¹, 3283 cm⁻¹ illustrated in FIGS. 2A and 2B. No substantial change in the IR spectrum was observed after 6 month storage indicating the stability of the crystalline Form H of Risdiplam.

In yet another embodiment, the crystalline Form H of Risdiplam prepared by the present process was characterized by a differential calorimetry thermogram (DSC) comprising two endotherms (i) a broad endothermic peak at 242-248° C. and an endothermic peak with an onset at 280.55° C. and a peak at temperature of 286° C. as illustrated in FIG. 3A-3C.

In yet another embodiment, the present invention discloses a pharmaceutical composition comprising of crystalline Form H of Risdiplam prepared by the present process together with pharmaceutically acceptable excipient.

The pharmaceutical composition may be formulated in solid form which include but not limited to powders, tablets, capsules, granules, pellets and the like; the liquid forms include but not limited to solutions, suspensions and emulsions; parenteral forms. The pharmaceutically acceptable carriers include but not limited to diluents, binders, preservatives, disintegrating agents, encapsulating agents, solubilizing agents, surfactants, flavorants and colorants.

In yet another embodiment, the crystalline Form H of Risdiplam obtained in the present invention is used as a medicament for treatment of spinal muscular atrophy (SMA).

In another embodiment, the present invention relates to a method of treating spinal muscular atrophy (SMA) by administering therapeutically effective amount to a subject in need thereof the crystalline Form H of Risdiplam prepared by the present process.

In another embodiment, the crystalline Form H of Risdiplam is stable up to a period of 6 months as illustrated by IR spectrum in FIG. 2.

The following examples, which include preferred embodiments, will serve to illustrate the practice of this invention, it being understood that the particulars shown are by way of examples and for purpose of illustrative discussion of preferred embodiments of the invention.

### Example-1: Preparation of Crystalline Risdiplam Form H

Process: To a mixture of Acetic acid (2.0 ml) and Methanol (20.0 ml) was added Risdiplam (2.0 g) under stirring at 30±5° C. so that it dissolves completely and precipitated out as crystalline solid which was filtered, washed with Methanol (4.0 ml) and dried to get title compound. Yield=1.8 g; % Yield=90.0%; Purity 99.54%.

### Example-2: Preparation of Crystalline Risdiplam Form H

To a mixture of Acetic acid (1.0 ml) and tertiary butanol (10.0 ml) was added Risdiplam (1.0 g) under stirring at 30±5° C. so that it dissolves completely and precipitated out as crystalline solid which was filtered, washed with tertiary butanol (2.0 ml) and dried to get title compound. Yield=0.82 g; % Yield=82.0%; purity 99.34%.

### Example 3: Comparative Table of the Present Risdiplam Form H and Form A Disclosed in WO2020079203A1

| | IR | | | | | DSC | TGA | Form |
|---|---|---|---|---|---|---|---|---|
| WO2020079203A1 | 848 | 885 | 939 | 1218 | | 298 | | Form-A |
| (Form A) | | | | | | | | |
| 011/DS- | 848 | 885 | 939 | 1217 | | 291.22 | 0.006% | Matches with |
| C/V/118/032 | | | | | | | | Form-A |
| (Form A | | | | | | | | |
| Prepared By | | | | | | | | |
| Harman) | | | | | | | | |
| 011/DS- | 834 | 880 | 926 | 1210 | 1227 | 286.14 | 0.172% | Matches with |
| C/IV/118/048 | | | | | | | | Form-H |
| 011/DS- | 834 | 880 | 926 | 1210 | 1227 | 284.9 | 0.025% | Matches with |
| C/IV/118/049 | | | | | | | | Form-H |
| 011/DS- | 834 | 880 | 926 | 1210 | 1227 | 286.11 | 0.047% | Matches with |
| C/IV/118/050 | | | | | | | | Form-H |
| 011/DS- | 834 | 880 | 926 | 1210 | 1226 | 283.82 | 0.18% | Matches with |
| C/V/138/005 | | | | | | | | Form-H |

### Example 4: Risdiplam Solubility Data

| Batch No | Water | Ethanol | Form |
|---|---|---|---|
| STD | <0.1 mg/ml | <1 mg/ml | |
| 011/DS-C/V/118/032 | 0.003 mg/ml | 1.3 mg/ml | Form-A |
| 011/DS-C/IV/118/048 | 0.006 mg/ml | 1.4 mg/ml | Form-H |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. A crystalline Form H of risdiplam of Formula-1 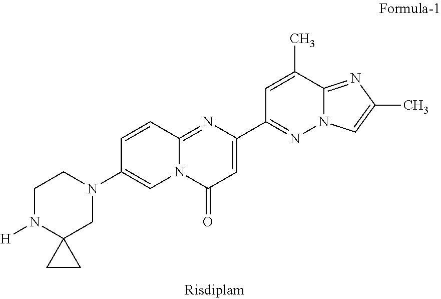 [SMILES: Cc1cn2nc(-c3cc(=O)n4cc(N5CCNC6(CC6)C5)ccc4n3)cc(C)c2n1]
wherein the crystalline Form H is characterized by
(i) an X-ray powder diffraction pattern containing at least one of the following 2 theta values (±0.2θ) at 5.94, 11.05, 15.41, 15.99, 17.23, 20.27, 23.592 24.74, 26.53, 27.07 2θ±0.2θ;
(ii) an infrared spectrum (IR) having peaks at about 834 cm⁻¹, about 880 cm⁻¹, about 926 cm⁻¹, about 1210 cm⁻¹ and about 1227 cm⁻¹, about 1674 cm⁻¹, 2824 cm⁻¹, and about 3283 cm⁻¹;
(iii) Aa broad endothermic peak at 242-248° C. and an endothermic peak with an onset at 280.55° C. and a peak at a temperature of about 286° C. as measured by differential scanning calorimetry (DSC).

2. The crystalline Form H of risdiplam, as claimed in claim 1, wherein the crystalline form is an anhydrous form with a water content of less than 2%.

3. The crystalline Form H of risdiplam as claimed in claim 2, wherein the water content ranges between 0.17% to 0.05% as determined by thermogravimetric analysis (TGA).

4. A process for preparing the crystalline Form H of risdiplam as claimed in claim 1, the process comprising:
a. adding risdiplam in a mixture of solvent(s);
b. stirring at a temperature ranging between 25° C. to 400° C.; and
c. precipitating a crystalline solid obtained in step b) followed by filtering, washing, and drying.

5. The process as claimed in claim 4, wherein the solvent comprises a polar solvent, a non-polar solvent, a protic solvent, an aprotic solvent or a mixtures thereof.

6. The process as claimed in claim 4, wherein the solvent comprises a lower C1-C4 alcohol, an acid, or a mixture thereof.

7. The process as claimed in claim 4, wherein the process is free of chromatographic purification.

8. A pharmaceutical composition comprising the crystalline Form H of risdiplam of as claimed in claim 1 and a pharmaceutically acceptable excipient.

9. (canceled)

10. A method of treating-spinal muscular atrophy (SMA) comprising administering the crystalline Form H of risdiplam as claimed in claim 1 in therapeutically effective amount to a subject in need thereof.

11. The process as claimed in claim 5, wherein the solvent comprises an alcohol, an acid, an ester, a hydrocarbon, or a mixture thereof.

12. The process as claimed in claim 4, wherein the solvent comprises a mixture of a lower C1-C4 alcohol and an acid.

13. The process as claimed in claim 12, wherein the lower C1-C4 alcohol comprises methanol and the acid comprises acetic acid.

14. The process as claimed in claim 4, wherein the solvent comprises a lower C1-C4 alcohol.

15. The process as claimed in claim 14, wherein the solvent comprises methanol.

16. The process as claimed in claim 4, wherein the solvent comprises an acid.

17. The process as claimed in claim 16 wherein the acid comprises acetic acid.

18. The process as claimed in claim 4, wherein the crystalline Form H of risdiplam is obtained in a yield of about more than 80%.

19. The process as claimed in claim 4, wherein the crystalline Form H of risdiplam is obtained having a purity in the range of 99.3% to 99.7%.
